(19) United States
(12) Patent Application Publication
(10) Pub. No.: US 2022/0163514 A1
(43) Pub. Date: May 26, 2022

(54) SYSTEM FOR CONDUCTING URINALYSIS

(71) Applicant: Vivosens Inc, San Francisco, CA (US)

(72) Inventors: Miray Tayfun, Istanbul (TR); Balim Bengisu Karacaoglu, Istanbul (TR); George Radman, Melbourne (AU)

(21) Appl. No.: 17/456,376

(22) Filed: Nov. 24, 2021

Related U.S. Application Data
(60) Provisional application No. 63/117,464, filed on Nov. 24, 2020.

(51) Int. Cl. G01N 33/52; B01L 3/00; A47K 13/24; G01N 21/78; G01N 33/543

(52) U.S. Cl. CPC G01N 33/521; B01L 3/5023; B01L 13/00; G01N 2021/7759; A47K 13/24; G01N 21/78; G01N 33/54388; B01L 3/50855

(57) ABSTRACT

A system for conducting urinalysis of urine is provided. The system includes a housing mountable on a urination device defining urination area; a dispenser mounted in the housing and being configured for holding a plurality of urine test strips (hereinafter “UTS”). Each one of the UTS includes at least one reacting area configured for a chemical reaction with a substance in the urine. The system further includes a detector configured for detecting at least one property of the reacting area and a shifting mechanism configured for extracting one UTS from the dispenser and moving the UTS to a urination position in which the UTS is disposed above the urination area, such that the reacting area is disposed along a urine path.

### FIELD OF INVENTION

The presently disclosed subject matter relates to a system for conducting urinalysis.

### BACKGROUND

Urinalysis is the physical, chemical, and microscopic examination of urine. It involves several tests to detect and measure various compounds that pass through the urine. When done on a frequent basis, urinalysis allows tracking changes in a person's body chemistry on a day-to-day basis.

### SUMMARY OF INVENTION

There is provided in accordance with an aspect of the presently disclosed subject matter a system for conducting urinalysis of urine. The system includes a housing mountable on a urination device defining urination area; a dispenser mounted in the housing and being configured for holding a plurality of urine test strips (hereinafter “UTS”). Each one of the UTS includes at least one reacting area configured for a chemical reaction with a substance in the urine. The system further includes a detector configured for detecting at least one property of the reacting area and a shifting mechanism configured for extracting one UTS from the dispenser and moving the UTS to a urination position in which the UTS is disposed above the urination area, such that the reacting area is disposed along a urine path.

The shifting mechanism can be further configured for moving the UTS to a testing position in which the UTS is disposed at a location with respect to the detector facilitating thereby detection of the at least one property.

The urination device can be a seating toilet and the housing is a toilet seat.

The dispenser can be disposed on a first side of the perimeter of the urination area and the detector is disposed on a second side of the perimeter, and wherein the shifting mechanism includes an arm configured for holding the UTS and the shifting mechanism is configured for rotating the arm between the dispenser and the urination area.

The dispenser, the urination area and the detector can be disposed along a circular path, and wherein the shifting mechanism is configured for rotating the arm along the circular path.

The system can further include a waste compartment configured to hold used UTS, and wherein the shifting mechanism is configured to release used UTS into the waste compartment.

The system can further include an ejection aperture configured to allow access to used UTS, and wherein the shifting mechanism is configured to release used UTS into the ejection aperture.

The system can further include a controller for controlling operation of the shifting mechanism and the detector and a pressure sensor configured to detect when urine impinges upon the UTS, and wherein the controller is configured to control an amount of urine impinges upon the UTS.

The system can further include a disinfection device for cleaning the detector.

The UTS can include a holding portion and a reacting area, and wherein the holding portion has a narrower width than the width of the reacting area. The UTS can be a colorimetric test strip and includes a plurality of pads each of which being configured for color change in response to reaction with the substance.

The at least one property can be a color of the pads and wherein the detector includes an array of sensors, each of which is configured a color in one of the pads.

The UTS can be a lateral flow test strip and wherein the reacting area includes a pad portion and an indicator portion, and wherein the shifting mechanism is configured to hold the UTS in the urination position, such that the pad portion is disposed along the urine path, and wherein the shifting mechanism is configured to hold the UTS in the testing position, such that the indicator portion is aligned with the detector and wherein the at least one property is a presence of the indicator.

The system can further include a communication module for communication with a remote IoT device and wherein the controller is configured to be controlled by the remote IoT device.

The detector can be configured to obtain an image of the UTS, and the communication module is configured to send the image to the remote IoT device.

The system of can further include a server in communication with the remote IoT device, and wherein the server is configured to receive urinalysis data of one urine test and determine kind and schedule for future urinalysis tests in accordance with the urinalysis data.

The UTS can be configured to detect at least one substance selected from the group consisting of calcium, UTIs, specific gravity, nitrites, blood, glucose, selenium, C peptide, chromium, pH, creatine, zinc, urobilinogen, bilirubin, leukocytes, microalbumin, urea, aldosterone total alkalinity, magnesium, potassium, hardness, iron, copper, lead, nitrate, fluoride, residual chlorine, breast cancer-specific antigen, prostate cancer-specific antigen, human chorionic gonadotropin, dopamine, serotonin, cortisol, luteinizing hormone, vitamin C, vitamin B12, vitamin B7, vitamin B9, heavy metals, estrogens, androgens, melatonin, progesterone, Testosterone, Sodium, chloride cortisone, Malondialdehyde, Peroxide, 8-OHdG free T3, free T4 drug abuse tests, and combinations thereof.

### BRIEF DESCRIPTION OF THE DRAWINGS

In order to understand the disclosure and to see how it may be carried out in practice, embodiments will now be described, by way of non-limiting examples only, with reference to the accompanying drawings, in which:

FIG. 1 is a side perspective view of the system for conducting urinalysis in accordance with an example of the presently disclosed subject matter;

FIG. 2A is a partially exploded view of the system of FIG. 1 showing the shifting mechanism in a first position;

FIG. 2B is an enlarged view of the shifting mechanism of the system of FIG. 1;

FIG. 3 is a partially exploded view of the system of FIG. 1 showing the shifting mechanism in a second position;

FIG. 4 is a partially exploded view of the system of FIG. 1 showing the shifting mechanism in a third position;

FIG. 5 is an upper view of the system for conducting urinalysis in accordance with another example of the presently disclosed subject matter;

FIG. 6A is an upper view of a urine test strip in accordance with an example of the presently disclosed subject matter;

FIG. 6B is an upper view of a urine test strip in accordance with another example of the presently disclosed subject matter; and

FIG. 7 is a block diagram showing the disposition of the urine test strip of FIG. 6A, with respect to the detector.

### DETAILED DESCRIPTION OF EMBODIMENTS

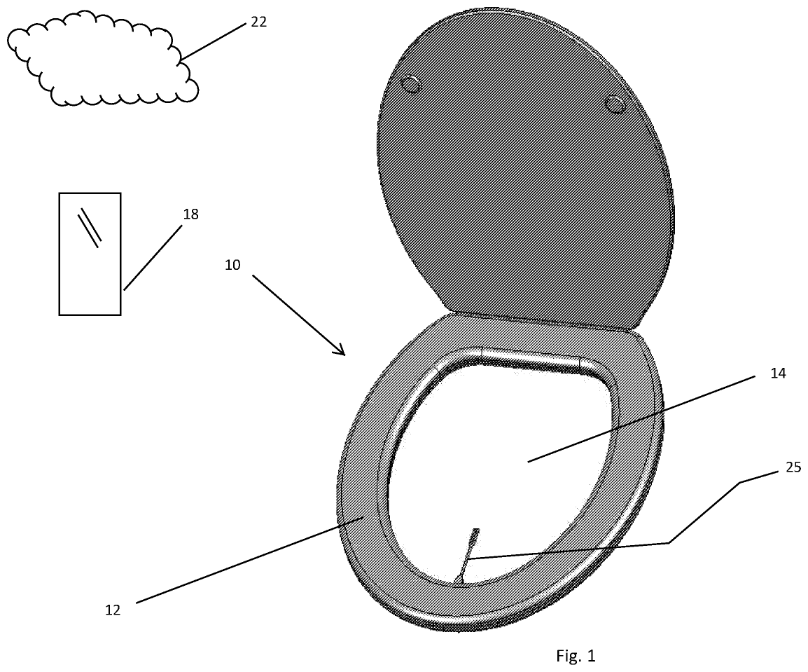

FIGS. 1-3 show a system 10 for conducting urinalysis of urine by using a urine test strip disposed along the urine path. The system 10 includes a housing 12 mountable on a urination device (not shown) defining urination area 14. According to the illustrated example, the urination device is a seating toilet, and the housing 12 is a toilet seat mounted on the toilet. According to other examples, the urination device can be a squat toilet, urinal, female urinal etc., and the housing can be a designated structure mounted on the urination device and defining a urination area 14.

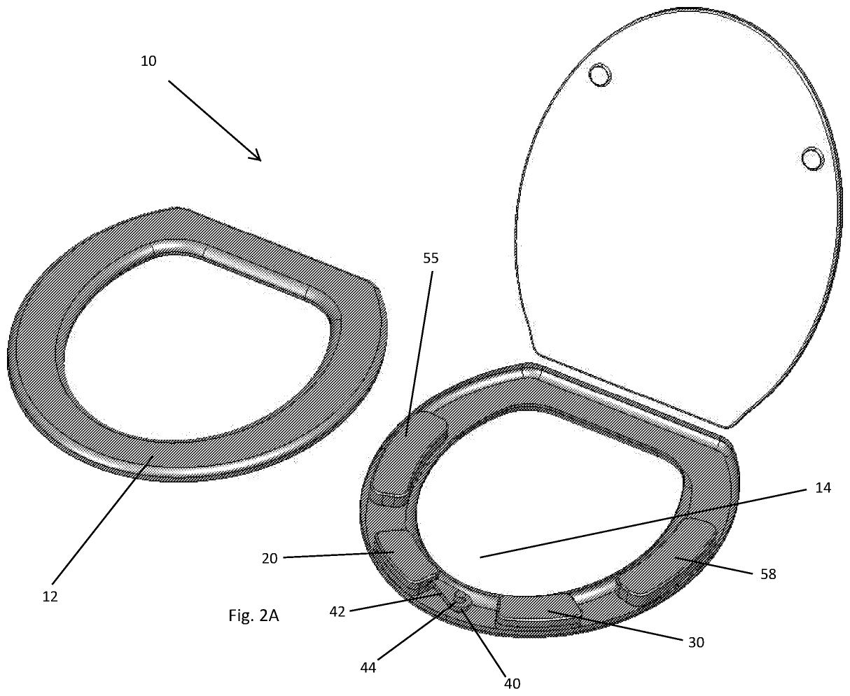

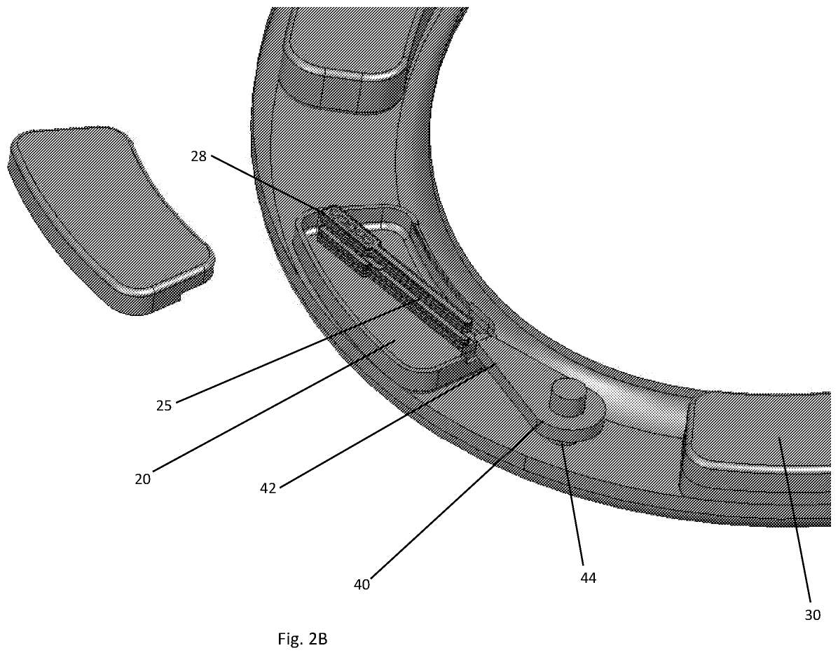

As shown in FIGS. 2A and 2B, the housing 12 includes a dispenser 20, which can be mounted inside the housing. The dispenser 20 is configured for holding a plurality of urine test strips (hereinafter “UTS”) 25, each of which having at least one reacting area 28 configured for a chemical reaction with a substance in the urine.

The UTS can be a lateral flow test having a reacting area 28 configured to detect the presence of a certain substance. Alternatively, the UTS can be a colorimetric test strip which includes one or more reacting area 28 in the form of pads. The reacting area 28 can be configured to react to various substances, such as calcium, UTIs, specific gravity, nitrites, blood, glucose, selenium, C peptide, chromium, pH, creatine, zinc, urobilinogen, bilirubin, leukocytes, microalbumin, urea, aldosterone total alkalinity, magnesium, potassium, hardness, iron, copper, lead, nitrate, fluoride, residual chlorine, breast cancer-specific antigen, prostate cancer-specific antigen, human chorionic gonadotropin, dopamine, serotonin, cortisol, luteinizing hormone, vitamin C, vitamin B12, vitamin B7, vitamin B9, heavy metals, estrogens, androgens, melatonin, progesterone, Testosterone, Sodium, chloride cortisone, Malondialdehyde, Peroxide, 8-OHdG free T3, free T4 drug abuse tests, and combinations thereof.

The housing 12 further includes a detector 30 configured for detecting at least one property of the reacting area 28. For example, the detector can be an optical sensor configured to detect a certain feature of the reacting area 28. For example, in case of lateral flow test, the detector 30 can be configured to detect the presence of an indicator on the reacting area 28, such as one or more lines indicating the presence of a certain substance in the urine or the lack of such substance.

Alternatively, in the case of a colorimetric test strip, the detector 30 can be configured to detect a predetermined color, a number of colors, or gray level on the reacting area 28. For example, the detector 30 can be configured to detect absorbance or can include a spectrophotometer for detecting certain colors. According to other examples, the detector 30 includes an imaging device configured for obtaining an image of the reacting area 28, and an image processing apparatus for analyzing the obtained image. Analyzing the obtained image can be carried out by utilizing known methods of analyzing a urinalysis, for example the method disclosed in PCT publication WO2019221676.

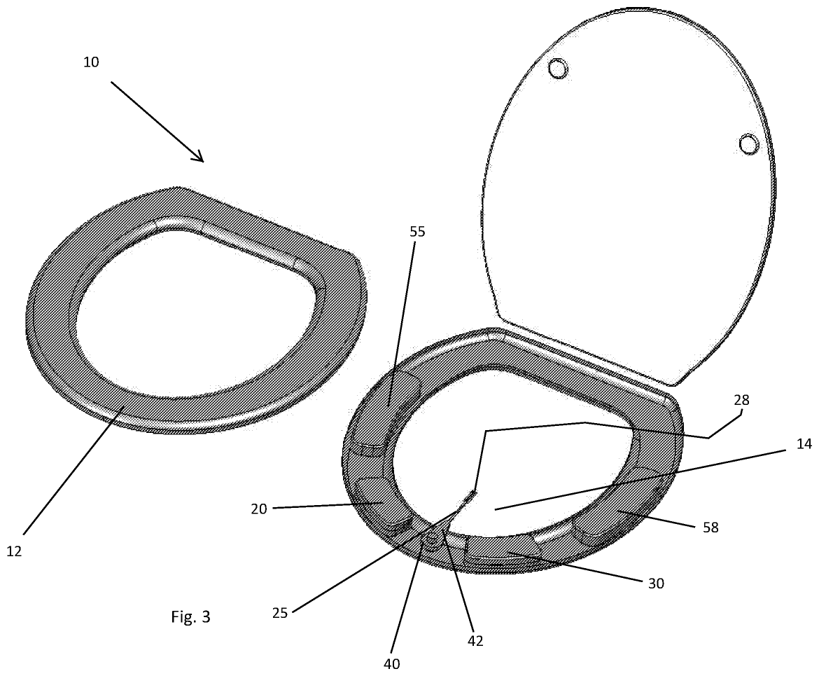

The system 10 further includes a shifting mechanism 40 configured for extracting one UTS 25 from the dispenser 20 and moving the UTS 25 to a urination position in which the UTS 25 is disposed above the urination area 14 such that the reacting area 28 is disposed along a urine path, as shown in FIG. 3.

The housing 12 can be provided with an opening (not shown) allowing removing of the dispenser 20, such that the latter can be periodically replenished.

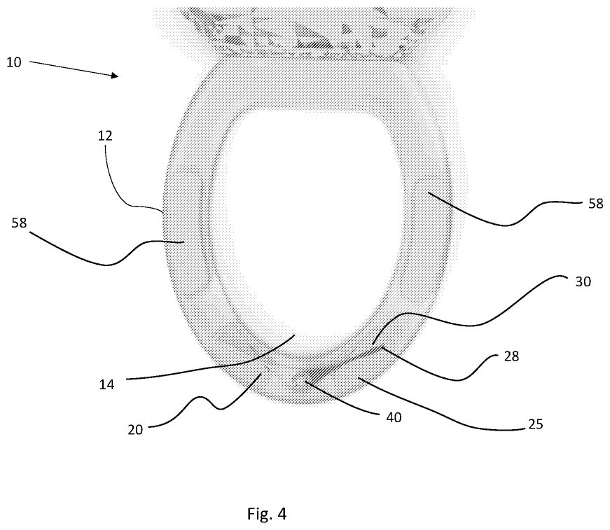

The shifting mechanism 40 can be further configured for moving the UTS 25 to a testing position in which the UTS 25 is disposed at a location with respect to the detector 30, facilitating thereby detection of properties of the reacting area 28, as described above. For example, the shifting mechanism 40 can be configured to shift the UTS 25 towards the detector 30 such that the reacting area 28 is disposed above the detector 30, as shown in FIG. 4.

According to the illustrated example, the dispenser 20 is disposed on a first side of the perimeter of the urination area 14, and the detector 30 is disposed on a second side of the perimeter of the urination area 14. The shifting mechanism 40 includes an arm 42 configured for holding the UTS 25, and the shifting mechanism includes a rotating motor 44 configured for rotating arm 42 between the dispenser 20 and urination area 14. Similarly, the arm 42 can be further rotated towards the detector 30, shifting the UTS to the testing position, as shown in FIG. 4.

As shown in FIG. 2B, the dispenser 20 can include a slit allowing the arm 42 to enter the dispenser and grab one of the UTS 25 therein. The dispenser 20 can be provided with a spring mechanism urging the upper most UTS 25 to the slit facilitating thereby engagement of the arm 42 with the UTS 25.

It would be appreciated by those skilled in the art that the shifting mechanism 40 may be configured to shift the UTS 25 from the dispenser 20 to the urination area 14 in other manners. For example, the dispenser 20 and the urination area 14 can be disposed along an axis and the shifting mechanism 40 can include a line conveyor configured to displace the UTS 25 from the dispenser 20 to the urination area 14.

The shifting mechanism 40 is further configured to release the UTS 25 after the completion of the test and detection by the detector 30. According to one example, the shifting mechanism 40 can be configured to rotate the UTS 25 back to the urination area 14 and to release the UTS 25 into the toilet. According to this example, the UTS 25 can be made from a biodegradable material or material which is decomposed and can be flashed in the toilet.

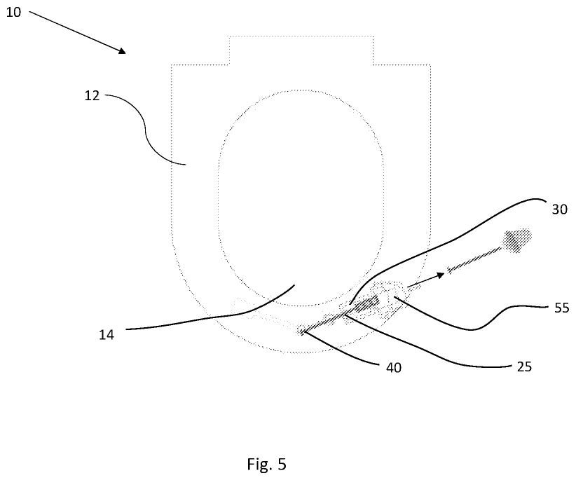

According to another example, as shown in FIG. 5, the system 10 can include a waste compartment 50 configured to hold a plurality of used UTSs 25. The waste compartment 50 can be located in close proximity to the detector 30, and the shifting mechanism 40 can be configured to shift the used UTS 25 into the waste compartment 50. According to an example, the waste compartment 50 is disposed along a straight line from the detector 30, such that when the shifting mechanism 40 releases the UTS 25, the latter falls into the waste compartment 50.

Alternatively, the detector 30 and the waste compartment 50 can be disposed along the same rotational path, such that the UTS is first shifted to the testing position above the detector 30. Further shifting of the UTS 25 along the same rotational path brings the latter into the waste compartment 50, where it can be released. The housing can be provided with an opening allowing removing of the waste compartment 50, such that the latter can be periodically emptied.

According to a further example, the housing 12 can include an ejection aperture (not shown) defined in close proximity of the detector 30. Upon completion of the test and detection of the UTS 25, the shifting mechanism 40 releases the UTS 25 into the ejection aperture, allowing the user to remove the used UTS 25.

The housing 12 further includes a power source, such as a battery 55, and a controller 58 for controlling the operation of the shifting mechanism 40 and the detector 30. The controller 58 may include a pressure sensor configured to detect when urine impinges upon the UTS 25. The controller 58 can be configured to control the amount of urine that reaches the UTS 25, for example, by initiating the shifting of the UTS 25 towards the detector 30 after a predetermined time since the urination starts. Alternatively, the pressure sensor can be utilized for detecting when urination stopped and to assess whether or not a sufficient amount of urine has reached the UTS 25.

The controller 58 can further include a processor for processing detection data received from the detector 30 and to determine health data in accordance with the detection data. According to an example, the controller 58 can further include a communication module, such as WiFi or Bluetooth transceiver, etc., for transmitting the detection or health data to a remote server 22 (as shown in FIG. 1), as explained herein after.

According to an example, the housing 12 may be provided with a disinfection device, such as, UV light bulb for cleaning the detector 30, or the ejection aperture, as described above.

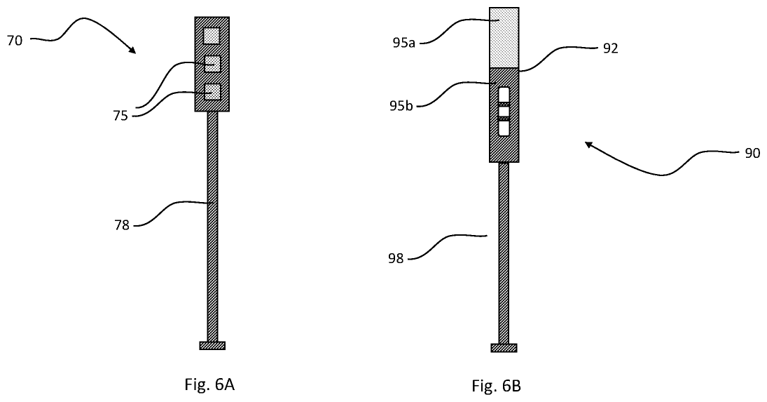

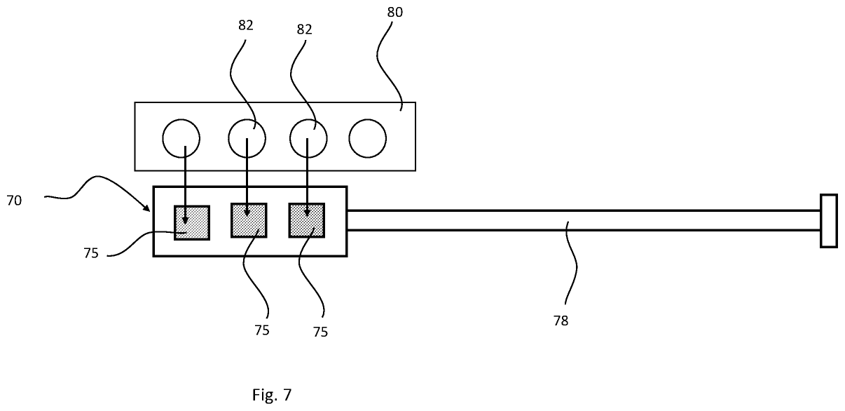

As shown in FIG. 6A, the UTS can be a colorimetric test strip 70 having a reacting area in the form of three or more pads 75, and a holding portion 78. The holding portion 78 is configured to be grasped by the shifting mechanism 40. As shown in the present example, the holding portion 78 is narrower relative to the reacting area, minimizing thereby the amount of urine impinging upon the holding portion 78. This way, the amount of urine entering areas around the detector 30 is reduced. According to this example, the urine reacts with the pads and causes the color of the pads to change depending on the presence of certain substances in the urine.

According to an example, each of the pads 75 can be configured to detect a different substance and to provide an indication with a predetermined color. Accordingly, as shown in FIG. 7, the detector 80 can be configured to detect each of the colors corresponding to the colors indicated by the pads 75. For example, the detector 80 can include an array of sensors 82, each of which is configured to detect one color. Thus, according to this example, the shifting mechanism 40 is configured to shift the UTS 70 towards the detector 80 such that the position of each of the pads 75 is aligned with the corresponding sensor 82.

According to another example, the UTS 90 can be a lateral flow test strip having a holding portion 98 and a reacting area 92. According to this example, reacting area 92 includes a pad portion 95a and an indicator portion 95b. Thus, the shifting mechanism 40 is configured such that when the UTS 90 is in the urination position, the pad portion 95a is disposed along the urine path, allowing the urine to react with the pad portion 95a. However, when the UTS 90 is shifted to the testing position, the shifting mechanism 40 is configured to dispose the UTS 90 such that the indicator portion 95b is aligned with the detector.

As shown in FIG. 1, according to an example, the system can be connected with IoT (Internet Of Things) devices 18, such as smartphones, watches, or other devices. According to this example, the detector can be configured to obtain an image of the UTS and data of the image is sent to an external IoT device 18. The external IoT device can include a microprocessor for processing the captured images and determining the results of the test. In some embodiments, the urinalysis system, which is connected to IoT devices (e.g. Smartphones) is configured to allows the user to control and operate the urinalysis system using such devices wirelessly. Alternatively, the urinalysis system can be operated using physical buttons that may be attached to any part of the toilet seat.

According to an example, the IoT device 18 can include a designated application with a user interface allowing the user to control various features of the system 10. For example, IoT device 18 can be configured to allow the user to alter the position of the arm 42. The arm 42 can be moved by the user to the appropriate location depending on the user's gender, height or preferences.

According to an example, a user can initiate a urinalysis test by clicking on the appropriate command or button on the manual button of a remote or IoT device 18. Alternatively, the IoT device can be configured to monitor the urine on a regular basis and provide the user with plans and wellness advice. Similarly, the urinalysis system can be configured for performing automated or manual tests depending upon the input of the user. Furthermore, the IoT device 18 can be connected to a server 22 which collects test data and provides medical recommendations in accordance with the results. The server 22 and the IoT device 18 can thus provide a testing schedule depending on the data received from the tests. In other words, in case data from a test indicates the existence of a certain substance in the urine, or the lack of a certain substance, the system server 22 and the IoT device 18 can be configured to determine a specific schedule for further tests, which can include specific time of the day, frequency of tests and or the kind of test required for monitoring health of the user. It would be appreciated that the toilet seat containing the urinalysis apparatus can be mounted on commonly available toilet bowls in the market.

It is noted that the known toilets that are capable of performing urinalysis are complex to build and use. One of the major problems with the existing smart toilets or toilet bowls is that they require a separate urine collection chamber. As a result, a toilet has to be redesigned and rebuilt or at the least, the toilet bowl has to be replaced with a new smart toilet bowl. The process of redesigning and rebuilding toilets is expensive. Hence, it is difficult to rebuild the toilets at a large scale. Further, cleaning such toilets with a urine collection chamber proves to be a difficult task for users. The existing smart toilets are also hard to operate.

The present invention aims to solve this problem. The present invention can be easily be integrated into most of the toilet bowls in the market. The present invention has an adjustable urination structure. The invention is easy to operate since it can be operated with IoT (Internet of Things) devices such as mobile phones, tabs, smartwatches, etc. The process is unlike the hospital or lab tests or manual at-home tests. The testing process is fully automated and the best part being, that no laboratory or clinical know-how is needed to perform or to analyze the test. The results can be analyzed and monitored on a mobile device. This will require no pre-requisite knowledge of technical know-how or clinical assistance for the user. With the cartilage disposing mechanism designed in the invention, urine strip waste can be disposed hygienically after the procedure.

Those skilled in the art to which the presently disclosed subject matter pertains will readily appreciate that numerous changes, variations, and modifications can be made without departing from the scope of the invention, mutatis mutandis.

1. A system for conducting urinalysis of urine, the system comprising:
a housing mountable on a urination device defining urination area;
a dispenser mounted in said housing and being configured for holding a plurality of urine test strips (hereinafter “UTS”), each one of said UTS includes at least one reacting area configured for a chemical reaction with a substance in the urine;
a detector configured for detecting at least one property of said reacting area;
a shifting mechanism configured for extracting one UTS from said dispenser and moving said UTS to a urination position in which said UTS is disposed above said urination area, such that said reacting area is disposed along a urine path.

2. The system of claim 1 wherein said shifting mechanism is further configured for moving said UTS to a testing position in which said UTS is disposed at a location with respect to said detector facilitating thereby detection of said at least one property.

3. The system of claim 1 wherein said urination device is a seating toilet and said housing is a toilet seat.

4. The system of claim 1 wherein the dispenser is disposed on a first side of the perimeter of the urination area and the detector is disposed on a second side of the perimeter, and wherein said shifting mechanism includes an arm configured for holding said UTS and said shifting mechanism is configured for rotating said arm between said dispenser and said urination area.

5. The system of claim 4 wherein the dispenser, the urination area and the detector are disposed along a circular path, and wherein said shifting mechanism is configured for rotating said arm along said circular path.

6. The system of claim 1 further comprises a waste compartment configured to hold used UTS, and wherein said shifting mechanism is configured to release used UTS into said waste compartment.

7. The system of claim 1 further comprises an ejection aperture configured to allow access to used UTS, and wherein said shifting mechanism is configured to release used UTS into said ejection aperture.

8. The system of claim 1 further comprises a controller for controlling operation of said shifting mechanism and said detector and a pressure sensor configured to detect when urine impinges upon said UTS, and wherein said controller is configured to control an amount of urine impinges upon said UTS.

9. The system of claim 1 further comprises a disinfection device for cleaning said detector.

10. The system of claim 1 wherein said UTS includes a holding portion and a reacting area, and wherein said holding portion has a narrower width than the width of said reacting area.

11. The system of claim 10 wherein said UTS is a colorimetric test strip and includes a plurality of pads each of which being configured for color change in response to reaction with said substance.

12. The system of claim 11 wherein said at least one property is a color of said pads and wherein said detector includes an array of sensors, each of which is configured a color in one of said pads.

13. The system of claim 10 wherein said UTS is a lateral flow test strip and wherein said reacting area includes a pad portion and an indicator portion, and wherein said shifting mechanism is configured to hold said UTS in said urination position, such that said pad portion is disposed along said urine path, and wherein said shifting mechanism is configured to hold said UTS in said testing position, such that said indicator portion is aligned with said detector and wherein said at least one property is a presence of said indicator.

14. The system of claim 8 further comprises a communication module for communication with a remote IoT device and wherein said controller is configured to be controlled by said remote IoT device.

15. The system of claim 14 wherein said detector is configured to obtain an image of said UTS, and said communication module is configured to send said image to said remote IoT device.

16. The system of claim 14 further comprising a server in communication with said remote IoT device, and wherein said server is configured to receive urinalysis data of one urine test and determine kind and schedule for future urinalysis tests in accordance with said urinalysis data.

17. The system of claim 1, wherein said UTS is configured to detect at least one substance selected from the group consisting of calcium, UTIs, specific gravity, nitrites, blood, glucose, selenium, C peptide, chromium, pH, creatine, zinc, urobilinogen, bilirubin, leukocytes, microalbumin, urea, aldosterone total alkalinity, magnesium, potassium, hardness, iron, copper, lead, nitrate, fluoride, residual chlorine, breast cancer-specific antigen, prostate cancer-specific antigen, human chorionic gonadotropin, dopamine, serotonin, cortisol, luteinizing hormone, vitamin C, vitamin B12, vitamin B7, vitamin B9, heavy metals, estrogens, androgens, melatonin, progesterone, Testosterone, Sodium, chloride cortisone, Malondialdehyde, Peroxide, 8-OHdG free T3, free T4 drug abuse tests, and combinations thereof.
